(19) United States
(12) Patent Application Publication
(10) Pub. No.: US 2007/0274999 A1
(43) Pub. Date: Nov. 29, 2007

(54) Method of treating disorders using platelet glycoprotein Ib alpha fusion polypeptides

(76) Inventor: Gray D. Shaw, Milton, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/804,652

(22) Filed: May 18, 2007

Related U.S. Application Data
(60) Provisional application No. 60/802,003, filed on May 18, 2006.

(51) Int. Cl. A61K 39/395; A61K 38/16

(52) U.S. Cl. 424/178.1; 514/8

(57) ABSTRACT

The present invention provides compositions and methods for treating or preventing thrombotic thrombocytopenic purpura, infective endocarditis, metastatic cancer, and sickle cell anemia disease.

### RELATED APPLICATIONS

This application claims priority to U.S. Ser. No. 60/802,003, filed May 18, 2006. The contents of this application are incorporated herein by reference in their entirety.

### FIELD OF THE INVENTION

The invention relates generally to compositions and methods for treating or preventing disorders, including vascular-associated disorders, and more particularly to methods for treating or preventing disorders using platelet glycoprotein Ibα-derived polypeptides.

### BACKGROUND OF THE INVENTION

Several physiological and pathological processes, such as vascular inflammation and repair response, involve adhesive interactions between various cell types normally found freely circulating in blood. Examples of such interactions include the interactions that can occur between platelets, leukocytes and the inner wall of blood vessels (i.e., the vascular endothelium). Under conditions of high fluid shear forces, platelets adhere to the endothelium via an interaction between the glycoprotein (GP) Ib-IX-V complex on their surface and von Willebrand factor (vWF) present on exposed vessel subendothelium. In addition, platelets adhering to the vascular endothelium can bind and capture freely circulating platelets via vWF-mediated tethering, enabling thrombus growth through successive layers of platelets. The GPIbα chain of the GPIb-IX-V complex can also facilitate the binding of α-thrombin to the platelet surface, enhancing the thrombin-mediated cleavage of GPV and protease-activated receptors (PARs).

In contrast, leukocytes can adhere to activated endothelium either directly or indirectly by first adhering to vWF-immobilized platelets. In both instances, leukocyte cell surface molecules that bind to either the selectin or integrin classes of adhesion receptors mediate these adhesion events. Leukocyte-platelet adhesion is thought to occur, in part, via interaction of the leukocyte surface integrin molecule MacI and the GPIb component of the platelet surface GPIb-IX-V complex.

### SUMMARY OF THE INVENTION

The invention provides various methods for treating, preventing, or reducing thrombotic thrombocytopenic purpura, infective endocarditis, sickle cell anemia disease, and metastatic cancer by administering glycoprotein-Ibα fusion polypeptides.

One method involves administering to a subject in need thereof a fusion polypeptide containing a first polypeptide operably linked to a second polypeptide, the first polypeptide containing at least a region of a glycoprotein Ibα polypeptide and the second polypeptide containing at least a region of an immunoglobulin polypeptide. Desirably, the first polypeptide that is part of the fusion polypeptide contains the amino acid sequence of SEQ ID NO: 1 (GPIb302/Ig), SEQ ID NO:2 (GPIb302/2A-Ig), SEQ ID NO:3 (GPIb302/2A-Ig), SEQ ID NO:4 (GPIb290-Ig), SEQ ID NO:5 (GPIb290/2V-Ig), or SEQ ID NO:6 (GPIb290/1A-Ig). Optionally, the fusion polypeptide is a multimeric polypeptide, such as a dimer.

Another method involves administering to a subject in need thereof an isolated fusion polypeptide containing a first polypeptide operably linked to a second polypeptide, where the first polypeptide contains an amino acid sequence with one to 20 amino acid substitutions, deletions or insertions relative to amino acids 1-290 of a human GPIbα protein sequence (SEQ ID NO:21) and where the first polypeptide has at least one of the following activities: (a) lower affinity binding to alpha thrombin relative to binding to alpha thrombin of a polypeptide containing the amino acid sequence of SEQ ID NO:22; (b) lower aggregation relative to aggregation of a polypeptide containing the amino acid sequence of SEQ ID NO:22; and (c) increased resistance to proteolysis than a polypeptide containing the amino acid sequence of SEQ ID NO:22. Optionally, the first polypeptide has no more than 15, 12, 10, or 5 substitutions, insertions, or deletions relative to the amino acid sequence of SEQ ID NO:21 or SEQ ID NO:22. The first polypeptide may bind with lower affinity to alpha thrombin relative to binding to alpha thrombin of a polypeptide containing the amino acid sequence of SEQ ID NO:21 or SEQ ID NO:22. Desirably, the first polypeptide contains at least one, two, or all of the amino acid substitutions Y276F, Y278F, Y279V, or a conservative variant thereof, relative to the amino acid sequence of SEQ ID NO:22. The polypeptide may contain the amino acid substitution C65S, K237V, or conservative variants thereof, relative to the amino acid sequence of SEQ ID NO:22. Optionally, aggregation of the first polypeptide is lowered relative to aggregation of a polypeptide comprising the amino acid sequence of SEQ ID NO:22. The first polypeptide is desirably more resistant to proteolysis than a polypeptide containing the amino acid sequence of SEQ ID NO:22. Optionally, the polypeptide contains an amino acid sequence with 1 to 10 amino acid substitutions, insertions, or deletions relative to amino acids 1-290 of SEQ ID NO:22, provided that the amino acid sequence includes 233V and 239V as well as the amino acid substitution Y276F. The amino acid sequence may also contain the amino acid substitution K237V, C65S, Y278F, Y279F, or combinations thereof. Desirably, the polypeptide binds with higher affinity to von Willebrand factor than a polypeptide containing the amino acid sequence of SEQ ID NO:21 or SEQ ID NO:22. Exemplary polypeptides contain the following combinations of amino acid substitutions in the amino acid sequence SEQ ID NO:22: Y276F; Y276F and K237V; Y276F and C65S; Y276F, Y278F, and Y279F; Y276F, Y278F, Y279F, and K237V; and Y276F, Y278F, Y279F, K237V, and C65S.

The invention also features a method of treating infective endocarditis by administering to a subject in need thereof a fusion polypeptide containing a first polypeptide with at least a region of a glycoprotein Ibα polypeptide and a second polypeptide containing at least a region of an immunoglobulin polypeptide. The fusion polypeptide interacts with a bacterium thereby preventing platelet aggregation.

A method of treating sickle cell anemia or thrombotic thrombocytopenia purpura by administering to a subject in need thereof a fusion polypeptide containing a first polypeptide with at least a region of a glycoprotein Ibα polypeptide and a second polypeptide with at least a region of an immunoglobulin polypeptide. The fusion polypeptide blocks von Willebrand Factor binding sites to prevent platelet adherence and in turn allowing for a reduced amount of restrained blood flow.

The invention features a method of treating metastatic cancer (e.g., metastatic breast cancer) by administering to a subject in need thereof a fusion polypeptide containing a first polypeptide with at least a region of a glycoprotein Ibα polypeptide and a second polypeptide containing at least a region of an immunoglobulin polypeptide. The fusion polypeptide reduces the ability of tumor cells to aggregate platelets thereby reducing their metastatic potential.

In all foregoing aspects of the invention, the subject being treating is desirably a mammal such as a human. Optionally, thrombocytopenic purpura or infective endocarditis is caused by Streptococci viridians and sickle cell anemia results from endothelium dysfunction in the subject. If desired, a second compound useful for treating a cardiovascular disorder is also administered to the subject including, for example, folic acid, an analgesic, an antibiotic (e.g., penicillin), hydroxyurea or analogs thereof, butyrate, arginine, Poloxamer 188, and sulphasalazine. Alternatively, the second compound is acetylsalicylic acid, heparin, a glycoprotein IIb/IIIa antagonist, clopidogrel, a P-selectin antagonist, a thrombin inhibitor, a thrombolytic enzyme, and/or low molecular weight heparin. If desired, the subject being treated for metastatic cancer is also treated with a second therapeutic regimen such as chemotherapy, radiotherapy, hormone therapy, or surgery. For example, the subject being treated with metastatic breast cancer is administered a second compound including, for example, abraxane, doxorubicin, pamidronate disodium, anastrozole, exemestane, cyclophosphamide, epirubicin, toremifene, letrozole, trastuzumab, megestrol, novaldex, paclitaxel, docetaxel, capecitabine, goserelin acetate, or zoledronic acid.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

The invention discloses various methods for treating, preventing, or reducing thrombotic thrombocytopenic purpura, infective endocarditis, sickle cell anemia disease, and metastatic cancer (e.g., metastatic breast cancer) using glycoprotein-Ibα polypeptides, which are preferably provided as fusion proteins. The fusion proteins, or nucleic acids encoding these fusion proteins, are incorporated into pharmaceutical compositions and administered to a subject. These polypeptides are useful for inhibiting adherence of platelets and leukocytes to biological tissues, such as the vascular endothelium, by inhibiting an interaction between a glycoprotein Ibα ligand (such as Von Willebrand Factor, Mac-1, P-selectin or thrombin) and a glycoprotein Ibα protein on the surface of a cell, such as a platelet. Inhibition of binding suppresses glycoprotein Ibα protein-mediated platelet aggregation and associated signal transduction in vivo. The glycoprotein Ibα protein-immunoglobulin fusion proteins may also be used to modulate the bioavailability of a glycoprotein Ibα protein cognate ligand. Inhibition of the glycoprotein Ibα protein ligand/glycoprotein Ibα-protein interaction are useful therapeutically for, inter alia, the treatment of vascular inflammation and other vascular disorders associated with platelet activation.

Glycoprotein Ibα-derived polypeptides with decreased binding to alpha thrombin, decreased aggregation and/or increased resistance to proteolysis are also useful as therapeutic agents in cases that would benefit from an inhibition of the binding of activated platelet cells to vWF on vascular cells. The protein variants of the invention with decreased thrombin binding advantageously result in decreased bleeding. Binding of thrombin to the platelet GPIbα receptor is necessary for clotting. Thrombin binding to a therapeutic soluble GPIb can augment bleeding in vivo by sequestering thrombin away from the platelet GPIbα receptor, thus reducing thrombin-induced platelet aggregation. Thus, the protein variants that show lower affinity for thrombin make the thrombin available to interact with the platelet Ibα receptor, which in turn promotes clotting.

In some embodiments, the methods are performed using polypeptides that comprise a region of a GPIbα polypeptide. Exemplary polypeptides, including fusion proteins containing regions of a GPIbα p fusion polypeptide, are disclosed in U.S. Pat. No. 6,991,796 and US patent application 20050089888, each of whose contents are incorporated herein by reference in their entirety. In some embodiments, the polypeptides comprise the amino acid sequence of SEQ ID NOs: 1-6, set forth below.

SEQ ID NO:1: GPIb302/Ig

(SEQ ID NO:1)

MPLLLLLLLLPSPLHPHPICEVSKVASHLEVNCDKRNLTALPPDLPKDTT
ILHLSENLLYTFSLATLMPYTRLTQLNLDRCELTKLQVDGTLPVLGTLDL
SHNQLQSLPLLGQTLPALTVLDVSFNRLTSLPLGALRGLGELQELYLKGN
ELKTLPPGLLTPTPKLEKLSLANNNLTELPAGLLNGLENLDTLLLQENSL
YTIPKGFFGSHLLPFAFLHGNPWLCNCEILYFRRWLQDNAENVYVWKQGV
DVKAMTSNVASVQCDNSDKFPVYKYPGKGCPTLGDEGDTDLYDYYPEEDT
EGDKVRATRTVVKFPTKARPHTCPPCPAPEALGAPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPVPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSvMHEALHNHYTQKSLSLSPGK
SEQ ID NO:2: GPIb302/2A-Ig

(SEQ ID NO:2)

MPLLLLLLLLPSPLHPHPICEVSKVASHLEVNCDKRNLTALPPDLPKDTT
ILHLSENLLYTFSLATLMPYTRLTQLNLDRCELTKLQVDGTLPVLGTLDL
SHNQLQSLPLLGQTLPALTVLDVSFNRLTSLPLGALRGLGELQELYLKGN
ELKTLPPGLLTPTPKLEKLSLANNNLTELPAGLLNGLENLDTLLLQENSL
YTIPKGFFGSHLLPFAFLHGNPWLCNCEILYFRRWLQDNAENVYVWKQGV
DVKAMTSNVASVQCDNSDKFPVYKYPGKGCPTLGDEGDTDLYDYYPEEDT
EGDKVAATATVVKFPTKARPHTCPPCPAPEALGAPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPVPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
SEQ ID NO:3: GPIb302/4X-Ig

(SEQ ID NO:3)

MPLLLLLLLLPSPLHPHPICEVSKVASHLEVNCDKRNLTALPPDLPKDTT
ILHLSENLLYTFSLATLMPYTRLTQLNLDRCELTKLQXTDGTLPVLGTLD
LSHNQLQSLPLLGQTLPALTVLDVSFNRLTSLPLGALRGLGELQELYLKG
NELKTLPPGLLTPTPKLEKLSLANNNLTELPAGLLNGLENLDTLLLQENS
LYTIPKGFFGSHLLPFAFLHGNPWLCNCEILYFRRWLQDNAENVYVWKQV
VDVKAVTSNVASVQCDNSDKFPVYKYPGKGCPTLGDEGDTDLYDYYPEED
TEGDKVAATATVVKFPTKARPHTCPPCPAPEALGAPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPVPIEKTISKAKGQPREPQVYTLP
PSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK
SEQ ID NO:4: GPIb290-Ig

(SEQ ID NO:4)

MPLLLLLLLLPSPLHPHPICEVSKVASHLEVNCDKRNLTALPPDLPKDTT
ILHLSENLLYTFSLATLMPYTRLTQLNLDRCELTKLQVDGTLPVLGTLDL
SHNQLQSLPLLGQTLPALTVLDVSFNRLTSLPLGALRGLGELQELYLKGN
ELKTLPPGLLTPTPKLEKLSLANNNLTELPAGLLNGLENLDTLLLQENSL
YTIPKGFFGSHLLPFAFLHNPWLCNCEILYFRRWLQDNAENVYVWKQGVD
VKANTSNVASVQCDNSDKFPVYKYPGKGCPTLGDEGDTDLYDYYPEEDTE
GDKVRPHTCPPCPAPEALGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPVPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
SEQ ID NO:5: GPIb290/2V-Ig

(SEQ ID NO: 5)

MPLLLLLLLLPSPLHPHPICEVSKVASHLEVNCDKRNLTALFPDLPKDTT
ILHLSENLLYTFSLATLMPYTRLTQLNLDRCELTKLQVDGTLPVLGTLDL
SHNQLQSLPLLGQTLPALTVLDVSFNRLTSLPLGALRGLGELQELYLKGN
ELKTLPPGLLTPTPKLEKLSLAINNNLTELPAGLLNGLENLDTLLLQENS
LYTIPKGFFGSHLLPFAFLHGNPWLCNCEILYFRRWLQDNAENVYVWKQV
VDVKAVTSNVASVQCDNSDKFPVYKYPGKGCPTLGDEGDTDLYDYYPEED
TEGDKVRPHTCPPCPAPEALGAPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPVPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGK
SEQ ID NO:6: GPIb290/1A-Ig

(SEQ ID NO: 6)

MPLLLLLLLLPSPLHPHPICEVSKVASHLEVNCDKRNLTALPPDLPKDTT
ILHLSENLLYTFSLATLMPYTRLTQLNLDRCELTKLQVDGTLPVLGTLDL
SHNQLQSLPLLGQTLPALTVLDVSFNRLTSLPLGALRGLGELQELYLKGN
ELKTLPPGLLTPTPKLEKLSLANNNLTELPAGLLNGLENLDTLLLQENSL
YTIPKGFFGSHLLPFAFLHGNPWLCNCEILYFRRWLQDNAENVYVWKQGV
DVAAMTSNVASVQCDNSDKFPVYKYPGKGCPTLGDEGDTDLYDYYPEEDT
EGDKVRPHTCPPCPAPEALGAPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPVPIEKTISKAKGQPREPQVYTLPPSREEMTKNQVSLT
CLVKGFYPSDIAVEWESNGQPENNYKTTPPVLDSDGSFFLYSKLTVDKSR
WQQGNVFSCSVMHEALHNHYTQKSLSLSPGK

DNA sequences encoding fusion polypeptides of SEQ ID NOs: 1-6 are disclosed below as sequences SEQ ID NOs: 15-20, respectively:

GPIb302-Ig nucleotide sequence

(SEQ ID NO:15)

atgcctctcctcctcttgctgctcctgctgccaagccccttacaccccca
ccccatctgtgaggtctccaaagtggccagccacctagaagtgaactgtg
acaagaggaatctgacagcgctgcctccagacctgccgaaagacacaacc
atcctccacctgagtgagaacctcctgtacaccttctccctggcaaccct
gatgccttacactcgcctcactcagctgaacctagataggtgcgagctca
ccaagctccaggtcgatgggacgctgccagtgctggggaccctggatcta
tcccacaatcagctgcaaagcctgcccttgctagggcagacactgcctgc
tctcaccgtcctggacgtctccttcaaccggctgacctcgctgcctcttg
gtgccctgcgtggtcttggcgaactccaagagctctacctgaaaggcaat
gagctgaagaccctgcccccagggctcctgacgcccacacccaagctgga
gaagctcagtctggctaacaacaacttgactgagctccccgctgggctcc
tgaatgggctggagaatctcgacacccttctcctccaagagaactcgctg
tatacaataccaaagggcttttttgggtcccacctcctgccttttgcttt
tctccacgggaacccctggttatgcaactgtgagatcctctattttcgtc
gctggctgcaggacaatgctgaaaatgtctacgtatggaagcaaggtgtg
gacgtcaaggccatgacctctaacgtggccagtgtgcagtgtgacaattc
agacaagtttcccgtctacaaatacccaggaaaggggtgccccacccttg
gtgatgaaggtgacacagacctatatgattactacccagaagaggacact
gagggcgataaggtgcgtgccacaaggactgtggtcaagttccccaccaa
agcgcggccgcacacatgcccaccgtgcccagcacctgaagccctggggg
caccgtcagtcttcctcttccccccaaaacccaaggacaccctcatgatc
tcccggacccctgaggtcacatgcgtggtggtggacgtgagccacgaaga
ccctgaggtcaagttcaactggtacgtggacggcgtggaggtgcataatg
ccaagacaaagccgcgggaggagcagtacaacagcacgtaccgtgtggtc
agcgtcctcaccgtcctgcaccaggactggctgaatggcaaggagtacaa
gtgcaaggtctccaacaaagccctcccagtccccatcgagaaaaccatct
ccaaagccaaagggcagccccgagaaccacaggtgtacaccctgccccca
tcccgggaggagatgaccaagaaccaggtcagcctgacctgcctggtcaa
aggcttctatcccagcgacatcgccgtggagtgggagagcaatgggcagc
cggagaacaactacaagaccacgcctcccgtgctggactccgacggcccc
ttcttcctctacagcaagctcaccgtggacaagagcaggtggcagcaggg
gaacgtcttctcatgctccgtgatgcatgaggctctgcacaaccactaca
cgcagaagagcctctccctgtctccgggtaaa
GPIb302/2A-Ig nucleotide sequence

SEQ ID NO:16)

atgcctctcctcctcttgctgctcctgctgccaagccccttacaccccca
ccccatctgtgaggtctccaaagtggccagccacctagaagtgaactgtg
acaagaggaatctgacagcgctgcctccagacctgccgaaagacacaacc
atcctccacctgagtgagaacctcctgtacaccttctccctggcaaccct
gatgccttacactcgcctcactcagctgaacctagataggtgcgagctca
ccaagctccaggtcgatgggacgctgccagtgctggggaccctggatcta
tcccacaatcagctgcaaagcctgcccttgctagggcagacactgcctgc
tctcaccgtcctggacgtctccttcaaccggctgacctcgctgcctcttg
gtgccctgcgtggtcttggcgaactccaagagctctacctgaaaggcaat
gagctgaagaccctgcccccagggctcctgacgcccacacccaagctgga
gaagctcagtctggctaacaacaacttgactgagctccccgctgggctcc
tgaatgggctggagaatctcgacacccttctcctccaagagaactcgctg
tatacaataccaaagggcttttttgggtcccacctcctgccttttgcttt
tctccacgggaacccctggttatgcaactgtgagatcctctattttcgtc
gctggctgcaggacaatgctgaaaatgtctacgtatggaagcaaggtgtg
gacgtcaaggccatgacctctaacgtggccagtgtgcagtgtgacaattc
agacaagtttcccgtctacaaatacccaggaaaggggtgccccacccttg
gtgatgaaggtgacacagacctatatgattactacccagaagaggacact
gagggcgataaggtggctgccacagcgactgtggtcaagttccccaccaa
agcgcggccgcacacatgcccaccgtgcccagcacctgaagccctggggg
caccgtcagtcttcctcttccccccaaaacccaaggacaccctcatgatc
tcccggacccctgaggtcacatgcgtggtggtggacgtgagccacgaaga
ccctgaggtcaagttcaactggtacgtggacggcgtggaggtgcataatg
ccaagacaaagccgcgggaggagcagtacaacagcacgtaccgtgtggtc
agcgtcctcaccgtcctgcaccaggactggctgaatggcaaggagtacaa
gtgcaaggtctccaacaaagccctcccagtccccatcgagaaaaccatct
ccaaagccaaagggcagccccgagaaccacaggtgtacaccctgccccca
tcccgggaggagatgaccaagaaccaggtcagcctgacctgcctggtcaa
aggcttctatcccagcgacatcgccgtggagtgggagagcaatgggcagc
cggagaacaactacaagaccacgcctcccgtgctggactccgacggcccc
ttcttcctctacagcaagctcaccgtggacaagagcaggtggcagcaggg
gaacgtcttctcatgctccgtgatgcatgaggctctgcacaaccactaca
cgcagaagagcctctccctgtctccgggtaaa
GPIb302/4X-Ignucleotide sequence

SEQ ID NO:17)

atgcctctcctcctcttgctgctcctgctgccaagccccttacaccccca
ccccatctgtgaggtctccaaagtggccagccacctagaagtgaactgtg
acaagaggaatctgacagcgctgcctccagacctgccgaaagacacaacc
atcctccacctgagtgagaacctcctgtacaccttctccctggcaaccct
gatgccttacactcgcctcactcagctgaacctagataggtgcgagctca
ccaagctccaggtcgatgggacgctgccagtgctggggaccctggatcta
tcccacaatcagctgcaaagcctgcccttgctagggcagacactgcctgc
tctcaccgtcctggacgtctccttcaaccggctgacctcgctgcctcttg
gtgccctgcgtggtcttggcgaactccaagagctctacctgaaaggcaat
gagctgaagaccctgcccccagggctcctgacgcccacacccaagctgga
gaagctcagtctggctaacaacaacttgactgagctccccgctgggctcc
tgaatgggctggagaatctcgacacccttctcctccaagagaactcgctg
tatacaataccaaagggcttttttgggtcccacctcctgccttttgcttt
tctccacgggaacccctggttatgcaactgtgagatcctctattttcgtc
gctggctgcaggacaatgctgaaaatgtctacgtatggaagcaagtggtg
gacgtcaaggccgtgacctctaacgtggccagtgtgcagtgtgacaattc
agacaagtttcccgtctacaaatacccaggaaaggggtgccccacccttg
gtgatgaaggtgacacagacctatatgattactacccagaagaggacact
gagggcgataaggtggctgccacagcgactgtggtcaagttccccaccaa
agcgcggccgcacacatgcccaccgtgcccagcacctgaagccctggggg
caccgtcagtcttcctcttccccccaaaacccaaggacaccctcatgatc
tcccggacccctgaggtcacatgcgtggtggtggacgtgagccacgaaga
ccctgaggtcaagttcaactggtacgtggacggcgtggaggtgcataatg
ccaagacaaagccgcgggaggagcagtacaacagcacgtaccgtgtggtc
agcgtcctcaccgtcctgcaccaggactggctgaatggcaaggagtacaa
gtgcaaggtctccaacaaagccctcccagtccccatcgagaaaaccatct
ccaaagccaaagggcagccccgagaaccacaggtgtacaccctgccccca
tcccgggaggagatgaccaagaaccaggtcagcctgacctgcctggtcaa
aggcttctatcccagcgacatcgccgtggagtgggagagcaatgggcagc
cggagaacaactacaagaccacgcctcccgtgctggactccgacggcccc
ttcttcctctacagcaagctcaccgtggacaagagcaggtggcagcaggg
gaacgtcttctcatgctccgtgatgcatgaggctctgcacaaccactaca
cgcagaagagcctctccctgtctccgggtaaa
GP1b290-Ig nucleotide sequence

(SEQ ID NO:18)

atgcctctcctcctcttgctgctcctgctgccaagccccttacaccccca
ccccatctgtgaggtctccaaagtggccagccacctagaagtgaactgtg
acaagaggaatctgacagcgctgcctccagacctgccgaaagacacaacc
atcctccacctgagtgagaacctcctgtacaccttctccctggcaaccct
gatgccttacactcgcctcactcagctgaacctagataggtgcgagctca
ccaagctccaggtcgatgggacgctgccagtgctggggaccctggatcta
tcccacaatcagctgcaaagcctgcccttgctagggcagacactgcctgc
tctcaccgtcctggacgtctccttcaaccggctgacctcgctgcctcttg
gtgccctgcgtggtcttggcgaactccaagagctctacctgaaaggcaat
gagctgaagaccctgcccccagggctcctgacgcccacacccaagctgga
gaagctcagtctggctaacaacaacttgactgagctccccgctgggctcc
tgaatgggctggagaatctcgacacccttctcctccaagagaactcgctg
tatacaataccaaagggcttttttgggtcccacctcctgccttttgcttt
tctccacgggaacccctggttatgcaactgtgagatcctctattttcgtc
gctggctgcaggacaatgctgaaaatgtctacgtatggaagcaaggtgtg
gacgtcaaggccatgacctctaacgtggccagtgtgcagtgtgacaattc
agacaagtttcccgtctacaaatacccaggaaaggggtgccccacccttg
gtgatgaaggtgacacagacctatatgattactacccagaagaggacact
gagggcgataaggtgcggccgcacacatgcccaccgtgcccagcacctga
agccctgggggcaccgtcagtcttcctcttccccccaaaacccaaggaca
ccctcatgatctcccggacccctgaggtcacatgcgtggtggtggacgtg
agccacgaagaccctgaggtcaagttcaactggtacgtggacggcgtgga
ggtgcataatgccaagacaaagccgcgggaggagcagtacaacagcacgt
accgtgtggtcagcgtcctcaccgtcctgcaccaggactggctgaatggc
aaggagtacaagtgcaaggtctccaacaaagccctcccagtccccatcga
gaaaaccatctccaaagccaaagggcagccccgagaaccacaggtgtaca
ccctgcccccatcccgggaggagatgaccaagaaccaggtcagcctgacc
tgcctggtcaaaggcttctatcccagcgacatcgccgtggagtgggagag
caatgggcagccggagaacaactacaagaccacgcctcccgtgctggact
ccgacggccccttcttcctctacagcaagctcaccgtggacaagagcagg
tggcagcaggggaacgtcttctcatgctccgtgatgcatgaggctctgca
caaccactacacgcagaagagcctctccctgtctccgggtaaa
GPIb290/2V-Ig nucleotide sequence

(SEQ ID NO:19)

atgcctctcctcctcttgctgctcctgctgccaagccccttacacccccac
cccatctgtgaggtctccaaagtggccagccacctagaagtgaactgtga
caagaggaatctgacagcgctgcctccagacctgccgaaagacacaacca
tcctccacctgagtgagaacctcctgtacaccttctccctggcaaccctg
atgccttacactcgcctcactcagctgaacctagataggtgcgagctcac
caagctccaggtcgatgggacgctgccagtgctggggaccctggatctat
cccacaatcagctgcaaagcctgcccttgctagggcagacactgcctgct
ctcaccgtcctggacgtctccttcaaccggctgacctcgctgcctcttgg
tgccctgcgtggtcttggcgaactccaagagctctacctgaaaggcaatg
agctgaagaccctgcccccagggctcctgacgcccacacccaagctggag
aagctcagtctggctaacaacaacttgactgagctccccgctgggctcct
gaatgggctggagaatctcgacacccttctcctccaagagaactcgctgt
atacaataccaaagggcttttttgggtcccacctcctgccttttgctttt
ctccacgggaacccctggttatgcaactgtgagatcctctattttcgtcg
ctggctgcaggacaatgctgaaaatgtctacgtatggaagcaagtggtgg
acgtcaaggccgtgacctctaacgtggccagtgtgcagtgtgacaattca
gacaagtttcccgtctacaaatacccaggaaaggggtgccccacccttgg
tgatgaaggtgacacagacctatatgattactacccagaagaggacactg
agggcgataaggtgcggccgcacacatgcccaccgtgcccagcacctgaa
gccctgggggcaccgtcagtcttcctcttccccccaaaacccaaggacac
cctcatgatctcccggacccctgaggtcacatgcgtggtggtggacgtga
gccacgaagaccctgaggtcaagttcaactggtacgtggacggcgtggag
gtgcataatgccaagacaaagccgcgggaggagcagtacaacagcacgta
ccgtgtggtcagcgtcctcaccgtcctgcaccaggactggctgaatggca
aggagtacaagtgcaaggtctccaacaaagccctcccagtccccatcgag
aaaaccatctccaaagccaaagggcagccccgagaaccacaggtgtacac
cctgcccccatcccgggaggagatgaccaagaaccaggtcagcctgacct
gcctggtcaaaggcttctatcccagcgacatcgccgtggagtgggagagc
aatgggcagccggagaacaactacaagaccacgcctcccgtgctggactc
cgacggccccttcttcctctacagcaagctcaccgtggacaagagcaggt
ggcagcaggggaacgtcttctcatgctccgtgatgcatgaggctctgcac
aaccactacacgcagaagagcctctccctgtctccgggtaaa
GPIb290/IA-Ig nucleotide sequence

(SEQ ID NO:20)

atgcctctcctcctcttgctgctcctgctgccaagccccttacacccccac
cccatctgtgaggtctccaaagtggccagccacctagaagtgaactgtga
caagaggaatctgacagcgctgcctccagacctgccgaaagacacaacca
tcctccacctgagtgagaacctcctgtacaccttctccctggcaaccctg
atgccttacactcgcctcactcagctgaacctagataggtgcgagctcac
caagctccaggtcgatgggacgctgccagtgctggggaccctggatctat
cccacaatcagctgcaaagcctgcccttgctagggcagacactgcctgct
ctcaccgtcctggacgtctccttcaaccggctgacctcgctgcctcttgg
tgccctgcgtggtcttggcgaactccaagagctctacctgaaaggcaatg
agctgaagaccctgcccccagggctcctgacgcccacacccaagctggag
aagctcagtctggctaacaacaacttgactgagctccccgctgggctcct
gaatgggctggagaatctcgacacccttctcctccaagagaactcgctgt
atacaataccaaagggcttttttgggtcccacctcctgccttttgctttt
ctccacgggaacccctggttatgcaactgtgagatcctctattttcgtcg
ctggctgcaggacaatgctgaaaatgtctacgtatggaagcaaggtgtgg
acgtcgcggccatgacctctaacgtggccagtgtgcagtgtgacaattca
gacaagtttcccgtctacaaatacccaggaaaggggtgccccacccttgg
tgatgaaggtgacacagacctatatgattactacccagaagaggacactg
agggcgataaggtgcggccgcacacatgcccaccgtgcccagcacctgaa
gccctgggggcaccgtcagtcttcctcttccccccaaaacccaaggacac
cctcatgatctcccggacccctgaggtcacatgcgtggtggtggacgtga
gccacgaagaccctgaggtcaagttcaactggtacgtggacggcgtggag
gtgcataatgccaagacaaagccgcgggaggagcagtacaacagcacgta
ccgtgtggtcagcgtcctcaccgtcctgcaccaggactggctgaatggca
aggagtacaagtgcaaggtctccaacaaagccctcccagtccccatcgag
aaaaccatictccaaagccaaagggcagccccgagaaccacaggtgtaca
ccctgcccccatcccgggaggagatgaccaagaaccaggtcagcctgacc
tgcctggtcaaaggcttctatcccagcgacatcgccgtggagtgggagag
caatgggcagccggagaacaactacaagaccacgcctcccgtgctggact
ccgacggccccttcttcctctacagcaagctcaccgtggacaagagcagg
tggcagcaggggaacgtcttctcatgctccgtgatgcatgaggctctgca
caaccactacacgcagaagagcctctccctgtctccgggtaaa

Other suitable polypeptides include amino acid sequences with one to 15 amino acid substitutions, deletions or insertions relative to amino acids in the region between, and including, amino acids 65-279 of a naturally occurring human GPIbα protein sequence shown in SEQ ID NO:21, or of the variant GPIb2V, whose amino acid sequence is shown in SEQ ID NO:22. In one or more embodiments, the polypeptide has one or more of the following activities: (i) lower affinity binding to alpha thrombin relative to binding to alpha thrombin of a polypeptide that includes the amino acid sequence of human SEQ ID NO:21 or SEQ ID NO:22; (ii) lower aggregation relative to aggregation of a polypeptide that includes the amino acid sequence of SEQ ID NO:21 or SEQ ID NO:22; or (iii) increased resistance to proteolysis relative to a polypeptide that includes the amino acid sequence of SEQ ID NO:21 or SEQ ID NO:22.

While not wishing to be bound by theory, it is believed that these properties are based on the results of substitutions at targeted regions in the polypeptide sequence of the GPIb2V variant (SEQ ID NO:22). The change from cysteine to serine residue at position 65 (i.e., C65S) leads to inhibition of aggregation of the GPIbα molecule, in particular during a recombinant production process. The tyrosine residues found at positions 276, 278, and 279 in the wild-type sequence are normally posttranslationally modified to sulfotyrosine, which creates an anionic electrostatic interaction with alpha thrombin. Selective elimination of these tyrosine residues by converting them to phenylalanine (i.e., Y276F, Y278F, and Y279F substitutions) reduces binding to alpha thrombin (Marchese et al., J. Biol. Chem. 270:9571-78), while retaining the desired binding to vWF. A lysine to valine substitution at position 237 will prevent proteolysis at this site during the recombinant production process.

The amino acid sequence of SEQ ID NO: 21, a 290 amino acid sequence fragment of a naturally occurring human glycoprotein Ibα chain, is set forth below:

(SEQ ID NO:21)

HPICEVSKVASHLEVNCDKRNLTALPPDLPKDTTILHLSENLLYTFSLATL
MPYTRLTQLNLDRCELTKLQVDGTLPVLGTLDLSHNQLQSLPLLGQTLPA
LTVLDVSFNRLTSLPLGALRGLGELQELYLKGNELKTLPPGLLTPTPKLE
KLSLANNNLTELPAGLLNGLENLDTLLLQENSLYTIPKGFFGSHLLPFAF
LHGNPWLCNCEILYFRRWLQDNAENVYVWKQGVDVKANTSNVASVQCDNS
DKFPVYKYPGKGCPTLGDEGDTDLYDYYPEEDTEGDKVR

The amino acid sequence of the GPIb2V variant is provided below as SEQ ID NO:22. This variant is also discussed in US patent application publication number 20030091576, and its counterpart WO 02/063003, both of which are hereby incorporated by reference.

(SEQ ID NO:22)

HPICEVSKVASHLEVNCDKRNLTALPPDLPKDTTILHLSENLLYTFSLATL
MPYTRLTQLNLDRCELTKLQVDGTLPVLGTLDLSHNQLQSLPLLGQTLPA
LTVLDVSFNRLTSLPLGALRGLGELQELYLKGNELKTLPPGLLTPTPKLE
KLSLANNNLTELPAGLLNGLENLDTLLLQENSLYTIPKGFFGSHLLPFAF
LHGNPWLCNCEILYFRRWLQDNAENVYVWKQVVDVKAVTSNVASVQCDNS
DKFPVYKYPGKGCPTLGDEGDTDLYDYYPEEDTEGDKVR

In some embodiments, the polypeptide has no more than 12 substitutions, insertions, or deletions relative to the amino acid sequence of SEQ ID NO:21 or SEQ ID NO:22. For example, it may have 10, 8, 7, 6, 5 or fewer substitutions, insertions, or deletions relative to the amino acid sequence of SEQ ID NO:21 or SEQ ID NO:22. In some embodiments, the substitutions, insertions, or deletions are in the region between amino acids 65 and 279, inclusive.

In some embodiments, the polypeptide binds with lower affinity to alpha thrombin relative to binding to alpha thrombin of a polypeptide that includes the amino acid sequence of SEQ ID NO:21 or SEQ ID NO:22. Examples of polypeptides that show reduced binding are polypeptides that include one, two, or three of the amino acid substitutions Y276F, Y278F, Y279V, or a conservative variant thereof, relative to the amino acid sequence of SEQ ID NO:21 or SEQ ID NO:22.

In some embodiments aggregation of the polypeptide is lowered relative to aggregation of a polypeptide that includes the amino acid sequence of SEQ ID NO:21 or SEQ ID NO:22. In some embodiments, decreased aggregation is observed during synthesis of the polypeptide in a cell. An example of a polypeptide that shows decreased aggregation is a polypeptide that includes the amino acid substitution C65S, or a conservative variant thereof, relative to the amino acid sequence of SEQ ID NO:21 or SEQ ID NO:22.

In some embodiments, the polypeptide is more resistant to proteolysis than a polypeptide that includes the amino acid sequence of SEQ ID NO:21 or SEQ ID NO:22. An example of such a polypeptide is a polypeptide includes the amino acid substitution K237V, or a conservative variant thereof, relative to the amino acid sequence of SEQ ID NO:21 or SEQ ID NO:22.

In some embodiments, the polypeptide includes an amino acid sequence with one to 10 amino acid substitutions, insertions, or deletions relative to amino acids 1-290 of SEQ ID NO:21 or SEQ ID NO:22, provided that at least one of the amino acid substitutions is C65S, K237V, Y276F Y278F, Y279F, K237V.

Examples of suitable polypeptides are those that have the following amino acid sequence substitutions relative to the amino acid sequence of SEQ ID NO:21 or SEQ ID NO:22: Y276F, Y276F K237V, Y276F C65S, Y276F Y278F Y279F, Y276F Y278F Y279F K237V and Y276F Y278F Y279F K237V C65S.

The invention also includes polypeptides with one or more, e.g., 2, 3, 5, 6, 8, 10, 15 or more amino acid substitutions in polypeptides derived from glycoprotein Ibα sequences in addition to those corresponding to SEQ ID NO:21 and SEQ ID NO:22. These include, GPIb302 (SEQ ID NO:7), GPIb302/2A (SEQ ID NO:8) GPIb/4× (SEQ ID NO:9), GPIb290 (SEQ ID NO: 10), GBIb290/2V (SEQ ID NO: 11) and GBIb290/1A (SEQ ID NO: 12).

(SEQ ID NO:7)

HPICEVSKVASHLEVNCDKRNLTALPPDLPKDTTILHLSENLLYTFSLATL
MPYTRLTQLNLDRCELTKLQVDGTLPVLGTLDLSHNQLQSLPLLGQTLPA
LTVLDVSFNRLTSLPLGALRGLGELQELYLKGNELKTLPPGLLTPTPKLE
KLSLANNNLTELPAGLLNGLENLDTLLLQENSLYTIPKGFFGSHLLPFAF
LHGNPWLCNCEILYFRRWLQDNAENVYVWKQGVDVKAMTSNVASVQCDNS
DKFPVYKYPGKGCPTLGDEGDTDLYDYYPEEDTEGDKVRATRTVVKFPTK
A

(SEQ ID NO:8)

HPICEVSKVASHLEVNCDKRNLTALPPDLPKDTTILHLSENLLYTFSLAT
LMPYTRLTQLNLDRCELTKLQVDGTLPVLGTLDLSHNQLQSLPLLGQTLP
ALTVLDVSFNRLTSLPLCALRGLGELQELYLKGNELKTLPPGLLTPTPKL
EKLSLANNNLTELPAGLLNGLENLDTLLLQENSLYTIPKGFFGSHLLPFA
FLHGNPWLCNCEILYFRRWLQDNAENVYVWKQGVDVKAMTSNVASVQCDN
SDKFPVYKYPGKGCPTLGDEGDTDLYDYYPEEDTEGDKVAATATVVKFPT
KA

(SEQ ID NO:9)

HPICEVSKVASHLEVNCDKRNLTALPPDLPKDTTILHLSENLLYTFSLAT
LMPYTRLTQLNLDRCELTKLQVDGTLPVLGTLDLSHNQLQSLPLLGQTLP
ALTVLDVSFNRLTSLPLGALRGLGELQELYLKGNELKTLPPGLLTPTPKL
EKLSLANNNLTELPAGLLNGLENLDTLLLQENSLYTIPKGFFGSHLLPFA
FLHGNPWLCNCEILYFRRWLQDNAENVYVWKQVVDVKAVTSNVASVQCDN
SDKFPVKYPGKGCPTLGDEGDTDLYDYYPEEDTEGDKVAATATVVKFPTK
A

(SEQ ID NO:10)

HPICEVSKVASHLEVNCDKRNLTALPPDLPKDTTILHLSENLLYTFSLAT
LMPYTRLTQLNLDRCELTKLQVDGTLPVLGTLDLSHNQLQSLPLLGQTLP
ALTVLDVSFNRLTSLPLGALRGLGELQELYLKGNELKTLPPGLLTPTPKL
EKLSLANNNLTELPAGLLNGLENLDTLLLQENSLYTIPKGFFGSHLLPFA
FLHGNPWLCNCEILYFRRWLQDNAENVYVWKQGVDVKAMTSNVASVQCDN
SDKFPVYKYPGKGCPTLGDEGDTDLYDYYPEEDTEGDKVR

(SEQ ID NO:11)

HPICEVSKVASNLEVNCDKRNLTALPPDLPKDTTILHLSENLLYTFSLAT
LMPYTRLTQLNLDRCELTKLQVDGTLPVLGTLDLSHNQLQSLPLLGQTLP
ALTVLDVSFNRLTSLPLGALRGLGELQELYLKGNELKTLPPGLLTPTPKL
EKLSLANNNLTELPAGLLNGLENLDTLLLQENSLYTIPKGFFGSHLLPFA
FLHGNPWLCNCEILYFRRWLQDNAENVYVWKQVVDVKAVTSNVASVQCDN
SDKFPVYKYPGKGCPTLGDEGDTDLYDYYPEEDTEGDKVR

(SEQ ID NO:12)

HPICEVSKVASHLEVNCDKRNLTALPPDLPKDTTILHLSENLLYTFSLAT
LMPYTRLTQLNLDRCELTKLQVDGTLPVLGTLDLSHNQLQSLPLLGQTLP
ALTVLDVSFNRLTSLPLGALRGLGELQELYLKGNELKTLPPGLLTPTPKL
EKLSLANNNLTELPAGLLNGLENLDTLLLQENSLYTIPKGFFGSHLLPFA
FLHGNPWLCNCEILYFRRWLQDNAENVYVWKQGVDVAAMTSNVASVQCDN
SDKFPVYKYPGKGCPTLGDEGDTDLYDYYPEEDTEGDKVR

Glycoprotein Variant Ibα Fusion Polypeptides

In various aspects the invention provides fusion proteins that include a first polypeptide containing at least a portion of a glycoprotein Ibα polypeptide variant operatively linked to a second polypeptide. As used herein, a glycoprotein Ibα “fusion protein” or “chimeric protein” includes at least a portion of a glycoprotein Ibα polypeptide variant operatively linked to a non-glycoprotein Ibα polypeptide. An “glycoprotein Ibα polypeptide” or “glycoprotein Ibα polypeptide variant” refers to a polypeptide having an amino acid sequence corresponding to at least a portion of a glycoprotein Ibα polypeptide, whereas a “non-glycoprotein Ibα polypeptide” refers to a polypeptide having an amino acid sequence corresponding to a protein that is not substantially homologous to the glycoprotein Ibα-protein, e.g., a protein that is different from the glycoprotein Ibα-polypeptide or fragment and that is derived from the same or a different organism. Within a glycoprotein Ibα fusion protein the glycoprotein Ibα polypeptide can correspond to all or a portion of an Ibα-protein.

In one embodiment, a glycoprotein Ibα fusion protein comprises at least one biologically active portion of a glycoprotein Ibα-protein. In another embodiment, a glycoprotein Ibα fusion protein comprises at least two biologically active portions of a glycoprotein Ibα protein. In yet another embodiment, a glycoprotein Ibα-fusion protein comprises at least three biologically active portions of a glycoprotein Ibα-protein. Within the fusion protein, the term “operatively linked” is intended to indicate that the first and second polypeptides are linked in a manner that allows for at least one function associated with a glycoprotein Ibα polypeptide. When used to refer to nucleic acids encoding a glycoprotein Ibα-fusion polypeptide, the term operatively linked means that a nucleic acid encoding the glycoprotein Ibα polypeptide and the non-glycoprotein Ibα polypeptide are fused in-frame to each other. The non-glycoprotein Ibα polypeptide can be fused to the N-terminus or C-terminus of the glycoprotein Ibα polypeptide.

In a further embodiment, the glycoprotein Ibα fusion protein may be linked to one or more additional moieties. For example, the glycoprotein Ibα fusion protein may additionally be linked to a GST fusion protein in which the glycoprotein Ibα fusion protein sequences are fused to the C-terminus of the GST (i.e., glutathione S-transferase) sequences. Such fusion proteins can facilitate the purification of glycoprotein Ibα fusion protein.

In another embodiment, the fusion protein includes a heterologous signal sequence (i.e., a polypeptide sequence that is not present in a polypeptide encoded by a glycoprotein Ibα nucleic acid) at its N-terminus. For example, the native glycoprotein Ibα signal sequence can be removed and replaced with a signal sequence from another protein. In certain host cells (e.g., mammalian host cells), expression and/or secretion of glycoprotein Ibα can be increased through use of a heterologous signal sequence. A representative signal sequence is MPLLLLLLLLPSPLHP (SEQ ID NO: 13). If desired, one or more amino acids can additionally be inserted between the first polypeptide moiety comprising the GPIbα moiety and the second polypeptide moiety.

A chimeric or fusion protein of the invention can be produced by standard recombinant DNA techniques. Nucleic acid sequences encoding GPIbα polypeptides, as well as the amino acid sequences of these polypeptides, from which variant GPIbα polypeptide variants are constructed are disclosed in WO02/063003, the contents of which are incorporated herein by reference in their entirety. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that encode a fusion moiety (e.g., an Fc region of an immunoglobulin heavy chain). A glycoprotein Ibα encoding nucleic acid can be cloned into such an expression vector such that the fusion moiety is linked in-frame to the immunoglobulin protein.

In various embodiments, the glycoprotein Ibα: fusion polypeptide includes the amino acid sequence of one or more of SEQ ID NOs: 1-6.

The first polypeptide, and/or nucleic acids encoding the first polypeptide, can be constructed using GPIbα-encoding sequences are known in the art and are described in, e.g. European Patent Application Publication No. 0 317 278 A2, and Lopez et al. 84:5615-19, 1987. Other sources for GPIbα: polypeptides and nucleic acids encoding GPIbα: polypeptides include GenBank Accession Nos. BAB12038 and AB038516, D85894 and BAA 12911, respectively (human sequences), and GenBank Accession No. AAC53320 and U91967, respectively, and are incorporated herein by reference in their entirety.

For example, DNA fragments coding for the polypeptide variant sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that encode a fusion moiety (e.g., an Fc region of an immunoglobulin heavy chain). A glycoprotein Ibα encoding nucleic acid can be cloned into such an expression vector such that the fusion moiety is linked in-frame to the immunoglobulin protein.

Glycoprotein Ibα fusion polypeptides may exist as oligomers, such as dimers or trimers. In some embodiments, the glycoprotein Ibα fusion polypeptide is a dimer.

In some embodiments, the GPIbα polypeptide moiety is provided as a variant GPIbα polypeptide having a mutation in the naturally-occurring GPIbα sequence (wild type) that results in higher affinity (relative to the non-mutated sequence) binding of the GPIbα polypeptide to a leukocyte cell surface molecule. For example, the mutant polypeptide may bind with higher affinity to von Willebrand factor (vWF). This increased reactivity, or hyperresponsiveness, can be assessed using low concentrations of ristocetin. Alternately, any other suitable means for determining the reactivity of the polypeptide with vWF can also be utilized to identify polypeptides which are “more” reactive with vWF, i.e. more reactive than naturally-occurring wild-type GPIbα. Examples of GPIbα polypeptide variants that bind with higher affinity to vWF include GPIbα variants that include sequence alterations in the hinge region of a GPIbα polypeptide. The hinge region is defined as the region including residues 220 to 310 and is reported to be a major binding site for vWF within the GPIbα polypeptide. Mutations in the hinge region include those at residue 233, which in the wild-type GPIbα encodes glycine. An example of a suitable substitution of is one in which the glycine at position 233 is replaced with valine (i.e., G233V). A second site for mutation at the hinge region is at residue 239, which in the wild-type GPIbα encodes methionine. A substitution of valine for methionine 239 is a representative substitution, but other amino acids can also be substituted. In addition, hinge region variants of GPIbα polypeptides suitable for use in a fusion polypeptide of the invention have mutations at residue both positions 233 and 239. (See, e.g., Dong et al., JBC 275:36 27663-27670 (2000)) Thus, the invention includes fusion proteins that have a substitution at position 239, e.g., an M239V substitution of a variant GPIbα polypeptide. Also within the invention is a fusion protein having a substitution at position 233, e.g., G233V, and a fusion protein that includes a variant GPIb a polypeptide with positions at both 233 and 239, e.g, a G233V and M239V substitution.

In some embodiments, the first polypeptide includes a full-length GPIbα polypeptide. Alternatively, the first polypeptide comprise less than full-length GPIbα polypeptide. For example the first polypeptide can be less than 600 amino acids in length, e.g., less than or equal to 500, 250, 150, 100, 50, or 25 amino acids in length.

In some embodiments, the second polypeptide is soluble. In some embodiments, the second polypeptide enhances the half-life, (e.g., the serum half-life) of the linked polypeptide. In some embodiments, the second polypeptide includes a sequence that facilitates association of the fusion polypeptide with a second GPIbα polypeptide. In some embodiments, the second polypeptide includes at least a region of an immunoglobulin polypeptide. Immunoglobulin fusion polypeptides are known in the art and are described in e.g., U.S. Pat. Nos. 5,516,964; 5,225,538; 5,428,130; 5,514,582; 5,714,147; and 5,455,165.

In some embodiments, the second polypeptide comprises a full-length immunoglobulin polypeptide. Alternatively, the second polypeptide comprises less than full-length immunoglobulin polypeptide, e.g., a heavy chain, light chain, Fab, Fab₂, Fv, or Fc. For example, in some embodiments the second polypeptide includes the heavy chain of an immunoglobulin polypeptide. In further embodiments, the second polypeptide includes the Fc region of an immunoglobulin polypeptide.

In one exemplary embodiment, the polypeptide is a dual function molecule having the sequence of SEQ ID NO: 23 and optionally, a signal peptide is also included (e.g.,

(SEQ ID NO:24)

MPLLLLLLLLPSPLHP
or

(SEQ ID NO:25)

MPLQLLLLLILLGPGNSLQLWDTWADEAEKALGPLLARDRR.

(SEQ ID NO:23)

QATEYEYLDYDFLPETEPPICEVSKVASHLEVNCDKRNLTALPPDLPKDT
TILHLSENLLYTFSLATLMPYTRLTQLNLDRCELTKLQVDGTLPVLGTLD
LSHNQLQSLPLLGQTLPALTVLDVSFNRLTSLPLGALRGLGELQELYLKG
NELKTLPPGLLTPTPKLEKLSLLANNNLTELPAGLLNGLENLDTLLLQEN
SLYTIPKGFFGSHLLPFAFLHGNPWLCNCEILYFRRWLQDNAENVYVWKQ
VVDVKAVTSNVASVQCDNSDKFPVYKYPGKGCPTLGDEGDTDLYDYYPEE
DTEGDKVR

A molecule that includes SEQ ID NO: 23 and the signal peptide of SEQ ID NO: 25, for example, may be produced in CHO cells as described herein. This dual function molecule blocks platelet GPIbα as well as selections.

In another aspect of the invention, the second polypeptide has less effector function than the effector function of an Fc region of a wild-type immunoglobulin heavy chain. Fc effector function includes for example, Fc receptor binding, complement fixation and T cell depleting activity (see, for example, U.S. Pat. No. 6,136,310). Methods of assaying T cell depleting activity, Fc effector function, and antibody stability are known in the art. In one embodiment the second polypeptide has low or no affinity for the Fc receptor. In an alternative embodiment, the second polypeptide has low or no affinity for complement protein Clq.

A representative second polypeptide sequence includes the amino acid sequence of SEQ ID NO: 14. This sequence includes an Fc region. Underlined amino acids are those that differ from the amino acid found in the corresponding position of the wild-type immunoglobulin sequence:

(SEQ ID NO:14)

HTCPPCPAPEALGAPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPVPIEKTISKAKGQPREPQVYTLPPSREEMTKNQVSLTCLVKGFY
PSDIAVEWESNGQPENNYKTTPPVLDSDGSFFLYSKLTVDKSRWQQGNVF
SCSVMHEALHNHYTQKSLSLSPGK

The GPIbα polypeptide variants can include, in addition to the designated substituted amino acid substitution, an alternative amino acid that fulfills the same function as the designated acid. In some embodiments, the alternative amino acid is related to the designated amino acid as a conservative amino acid substitution of the indicated amino acid. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Thus, in some embodiments, rather than replacing a tyrosine with a phenylalanine at position 276, 278, or 279 (Y276F, Y278F, or Y279F), the tyrosine residue can be replaced with tryptophan or histidine. Similarly, for the C65S variant, the cysteine may alternatively be replaced with glycine, asparagine, glutamine, threonine, or tyrosine. For the L237V substitution, the lysine can alternatively be replaced with alanine, leucine, isoleucine, proline, phenylalanine, methionine, or tryptophan.

The invention also includes a polypeptide whose sequence that are 80%, 85%, 90%, 95%, 98%, or 99% homologous to the sequence disclosed in SEQ ID NO: 1 or SEQ ID NO:2, provided that it includes one or more of the amino acid substations C65S, K237V, Y276F Y278F, Y279F, K237V. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (e.g., an identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman.

In addition to the GPIb sequences disclosed herein, it will be appreciated by those skilled in the art that DNA sequence polymorphisms that lead to changes in the amino acid sequences of GPIbα may exist within a population (e.g., the human population). Such genetic polymorphism in the GPIbα gene may exist among individuals within a population due to natural allelic variation. As used herein, the terms “gene” and “recombinant gene” refer to nucleic acid molecules comprising an open reading frame encoding a GPIbα protein, preferably a mammalian GPIbα protein. Such natural allelic variations can typically result in 1-5% variance in the nucleotide sequence of the GPIbα gene. Any and all such nucleotide variations and resulting amino acid polymorphisms in GPIbα that are the result of natural allelic variation and that do not alter the functional activity of GPIbα are intended to be within the scope of the invention.

Moreover, nucleic acid molecules encoding GPIbα proteins from other species, and thus that have a nucleotide sequence that differs from a disclosed sequence of are intended to be within the scope of the invention. Nucleic acid molecules corresponding to natural allelic variants and homologues of the GPIbα cDNAs can be isolated based on their homology to the human GPIbα nucleic acids disclosed herein using the human cDNAs, or a portion thereof, as a hybridization probe according to standard hybridization techniques under stringent hybridization conditions. For example, a soluble human cDNA can be isolated based on its homology to human membrane-bound GPIbα.

Accordingly, in another embodiment, an isolated nucleic acid molecule of the invention is at least 6 nucleotides in length and hybridizes under stringent conditions to GPIbα nucleic acid molecules. In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C.

In a second embodiment, a nucleic acid sequence that is hybridizable to a GPIbα nucleic acid sequence, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising a GPIbα nucleotide sequence, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc. Natl. Acad. Sci. USA 78: 6789-6792.

Another aspect of the invention pertains to vectors (including expression vectors) containing a nucleic acid encoding glycoprotein Ibα fusion polypeptides, or derivatives, fragments, analogs or homologs thereof. Recombinant expression vectors of the invention can be designed for expression of glycoprotein Ibα fusion polypeptides in prokaryotic or eukaryotic cells.

In another embodiment, the glycoprotein Ibα fusion polypeptide expression vector is a yeast expression vector. Examples of vectors for expression in yeast Saccharomyces cerevisiae are known in the art.

Alternatively, glycoprotein Ibα-fusion polypeptide can be expressed in insect cells using methods known in the art, e.g., with Baculovirus expression vectors.

In yet another embodiment, a nucleic acid of the invention is expressed in mammalian cells using a mammalian expression vector.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art.

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to a glycoprotein Ibα fusion polypeptide mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

A host cell can be any prokaryotic or eukaryotic cell. For example, glycoprotein Ibα fusion polypeptides can be expressed in bacterial cells such as E. coli, insect cells, yeast or mammalian cells (such as human, Chinese hamster ovary cells (CHO) or COS cells). Other suitable host cells are known to those skilled in the art.

Vector DNA can be introduced into prokaryotic or eukaryotic cells via conventional transformation or transfection techniques. Mammalian host cell, such as CHO or COS cells can be transfected with expression vectors to enable, via posttranslational modification, the generation of the sialyl Lewis^{X} epitope on the N-linked and O-linked glycans of glycoprotein Ibα fusion polypeptides. In the case of CHO cells this requires the co-expression of an α-1,3/1,4 fuicosyltranseferase (Kukowska-Latallo et al., Genes Dev. 4:1288-303, 1990) and Core2 beta-1,6-N-acetylglucosaminyltransferase enzymes. (Kumar et al., Blood 88:3872-79, 1996). The presence of the sialyl Lewis^{X} epitopes on the N-linked and O-linked glycans of glycoprotein Ibα fusion polypeptides will enhance the binding to selectins.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding glycoprotein Ibα fusion polypeptides or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) glycoprotein Ibα fusion polypeptides. Accordingly, the invention further provides methods for producing glycoprotein Ibα fusion polypeptides using the host cells of the invention. In one embodiment, the method comprises culturing the host cell of invention (into which a recombinant expression vector encoding a glycoprotein Ibα fusion polypeptide has been introduced) in a suitable medium such that glycoprotein Ibα fusion polypeptides are produced. In another embodiment, the method further comprises isolating glycoprotein Ibα fusion polypeptide from the medium or the host cell.

The fusion polypeptides may be isolated and purified in accordance with conventional conditions, such as extraction, precipitation, chromatography, affinity chromatography, electrophoresis or the like.

Chemical synthesis of polypeptides facilitates the incorporation of modified or unnatural amino acids, including D-amino acids and other small organic molecules. Replacement of one or more L-amino acids in a peptide with the corresponding D-amino acid isoforms can be used to increase the resistance of peptides to enzymatic hydrolysis, and to enhance one or more properties of biologically active peptides, i.e., receptor binding, functional potency or duration of action.

Introduction of covalent cross-links into a peptide sequence can conformationally and topographically constrain the polypeptide backbone. This strategy can be used to develop peptide analogs of the fusion polypeptides with increased potency, selectivity and stability. Because the conformational entropy of a cyclic peptide is lower than its linear counterpart, adoption of a specific conformation may occur with a smaller decrease in entropy for a cyclic analog than for an acyclic analog, thereby making the free energy for binding more favorable. Macrocyclization is often accomplished by forming an amide bond between the peptide N- and C-termini, between a side chain and the N- or C-terminus [e.g., with K₃Fe(CN)₆ at pH 8.5], or between two amino acid side chains. Disulfide bridges are also introduced into linear sequences to reduce their flexibility. Furthermore, the replacement of cysteine residues with penicillamine (Pen, 3-mercapto-(D) valine) has been used to increase the selectivity of some opioid-receptor interactions.

Pharmaceutical Compositions Including Glycoprotein Ibα Fusion Polypeptides or Nucleic Acids Encoding Same

The glycoprotein Ibα fusion proteins, or nucleic acid molecules encoding these fusion proteins, (also referred to herein as “Therapeutics” or “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The active agents disclosed herein can also be formulated as liposomes. Liposomes are prepared by methods known in the art. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents can be included in the composition, for example, sugars, polyalcohols such as mannitol and sorbitol; and sodium chloride. Prolonged absorption of the injectable compositions can be brought about by including in the composition an agent which delays absorption, for example, aluminum monostearate and gelatin.

Sterile injectable solutions can be prepared by incorporating the active compound (e.g., a glycoprotein Ibα fusion protein) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

In some embodiments, oral or parenteral compositions are formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration or by stereotactic injection. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Sustained-release preparations can be prepared, if desired. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The pharmaceutical compositions can be included in a container, pack, or dispenser together with instructions for administration.

As used herein a biological system is meant to include any system that comprises biological components, e.g., cells, proteins, carbohydrates, lipids or nucleic acids. The biological system can be an in vivo, ex vivo or in vitro system.

“Adherence” is meant to include any biological interaction of a leukocyte, e.g., rolling, firm attachments or specific interaction.

Inhibition of adherence of a blood cell or protein to a biological tissue can be measured using methods known in the art. For example, assays for detecting binding of glycoprotein Ibα to a biological tissue are described in Simon et al., J. Exp. Med. 192:193-204, 2000, and references cited therein. In various embodiments, binding of a GP Ibα fusion protein inhibits binding of a blood cell or protein to a biological tissue by at least 30%, 50%, 75%, 90%, 95%, 99% or 99.9%.

Adherence can also be assessed in condition of greater or less than physiological flow conditions, including static conditions and serial application of static and shear conditions. Adherence can be determined for example colormetrically, fluorometrically, by flow cytometry or using a parallel plate flow chamber assay.

If desired, the subject is also administered one or more of the following: a statin; acetylsalicylic acid (aspirin); heparin (including unfractionated or low-molecular weight heparins); glycoprotein IIb/IIIa antagonists; clopidogrel; P-selectin antagonists; thrombin inhibitors; and thrombolytic enzymes. Alternatively, the subject is administered an anti-cancer regimen including surgery, radiotherapy, chemotherapy or hormone therapy. Exemplary anti-cancer agents include abraxane, doxorubicin, pamidronate disodium, anastrozole, exemestane, cyclophosphamide, epirubicin, toremifene, letrozole, trastuzumab, megestrol, novaldex, paclitaxel, docetaxel, capecitabine, goserelin acetate, and zoledronic acid.

The subject can be e.g., any mammal, e.g., a human, a primate, mouse, rat, dog, cat, cow, horse, pig.

The therapeutics include, e.g.: (i) any one or more of the glycoprotein Ibα polypeptide and derivative, fragments, analogs and homologs thereof; (ii) antibodies directed against the glycoprotein Ibα polypeptides described in (i) and (iii) nucleic acids encoding a glycoprotein Ibα polypeptide, and derivatives, fragments, analogs and homologs thereof as described in (i) above.

Essentially any disorder that is etiologically linked to platelet activation is considered amenable to prevention or to treatment. The disorder can be, e.g., metastatic cancer (e.g., metastatic breast cancer), vascular inflammation; atherosclerosis; restenosis (e.g. angioplasty-related restenosis); and/or a condition associated with thrombotic disease, e.g., angina, (including stable angina and unstable angina) acute myocardial infarction, stroke, venous thrombosis or arterial thrombosis.

The invention provides a method of treating a disorder, the method comprising administering to a subject in need thereof a fusion polypeptide comprising a first polypeptide operably linked to a second polypeptide,

wherein the first polypeptide comprises at least a region of a glycoprotein Ibα polypeptide and the second polypeptide comprises at least a region of an immunoglobulin polypeptide,

wherein the disorder is selected from the group consisting of thrombotic thrombocytopenic purpura, infective endocarditis, metastatic cancer, and sickle cell anemia disease.

In some embodiments, the disorder is metastatic cancer. In some embodiments, the metastatic cancer is metastatic breast cancer.

In some embodiments, the method includes further comprising administering to the subject a second compound useful for treating a cardiovascular disorder.

In some embodiments, the second compound is folic acid, an analgesic, an antibiotic, hydroxyurea, butyrate, arginine, Poloxamer 188, or sulphasalazine.

In some embodiments, the disorder is sickle cell anemia.

In some embodiments, the antibiotic is penicillin.

In some embodiments, the second compound is hydroxyurea or analogs thereof.

In some embodiments, the second compound is acetylsalicylic acid, heparin, a glycoprotein IIb/IIIa antagonist, clopidogrel, a P-selectin antagonist, a thrombin inhibitor, or a thrombolytic enzyme.

In some embodiments, the subject is further administered a second therapeutic regimen useful for treating metastatic cancer.

In some embodiments, the second therapeutic regimen is radiotherapy, surgery, hormone therapy, or chemotherapy.

In some embodiments, the subject is further administered abraxane, doxorubicin, pamidronate disodium, anastrozole, exemestane, cyclophosphamide, epirubicin, toremifene, letrozole, trastuzumab, megestrol, novaldex, paclitaxel, docetaxel, capecitabine, goserelin acetate, or zoledronic acid.

In some embodiments, the first polypeptide is selected from the group consisting of SEQ ID NO:1 (GPIb302/Ig), SEQ ID NO:2 (GPIb302/2A-Ig), SEQ ID NO:3 (GPIb302/2A-Ig), SEQ ID NO:4 (GPIb290-Ig), SEQ ID NO:5 (GPIb290/2V-Ig), and SEQ ID NO:6 (GPIb290/1A-Ig).

In some embodiments, the fusion polypeptide consists essentially of the amino acid sequence of SEQ ID NO:5.

In some embodiments, the fusion polypeptide consists of the amino acid sequence of SEQ ID NO:5.

In some embodiments, the fusion polypeptide is a multimeric polypeptide.

In some embodiments, the multimeric polypeptide is a dimer.

In some embodiments, the polypeptide comprises an amino acid sequence with 1 to 10 amino acid substitutions, insertions, or deletions relative to amino acids 1-290 of SEQ ID NO:22, provided that the amino acid sequence includes 233V, 239V, and comprises the amino acid substitution Y276F.

In some embodiments, the amino acid sequence comprises the amino acid substitution K237V.

In some embodiments, the amino acid sequence further comprises the amino acid substitution C65S.

In some embodiments, the amino acid sequence further comprises the amino acid substitution Y278F or Y279F.

In some embodiments, the amino acid sequence further comprises the amino acid substitutions Y278F and Y279F.

In some embodiments, the further amino acid sequence further comprises the amino acid substitution K237V.

In some embodiments, the amino acid sequence further comprises the amino acid substitution C65S.

In some embodiments, the polypeptide binds with higher affinity to von Willebrand factor than a polypeptide comprising the amino acid sequence of SEQ ID NO:21 or SEQ ID NO:22.

In some embodiments, the polypeptide comprises one or more substitutions in the amino acid sequence SEQ ID NO:22, wherein the one or more substitutions is Y276F; Y276F K237V; Y276F C65S; Y276F Y278F Y279F; Y276F Y278F Y279F K237V; or Y276F Y278F Y279F K237V C65S.

In some embodiments, the subject is human.

In some embodiments, the infective endocarditis involves viridians streptococci microorganisms.

In some embodiments, the sickle cell anemia is a result of endothelium dysfunction.

In some embodiments, the method further comprises administering to the subject a compound selected from the group consisting of acetylsalicylic acid, heparin, a glycoprotein IIb/IIIa antagonist, clopidogrel, a P-selectin antagonist, a thrombin inhibitor, a thrombolytic enzyme, and low molecular weight heparin.

In some embodiments, the disorder is sickle cell anemia.

In some embodiments, the fusion polypeptide is administered in combination with folic acid, an analgesic, an antibiotic, hydroxyurea, butyrate, arginine, Poloxamer 188, or sulphasalazine.

In some embodiments, the antibiotic is penicillin.

In some embodiments, the fusion polypeptide is administered in combination with hydroxyurea or analogs thereof.

In some embodiments, the disorder is metastatic cancer.

In some embodiments, the metastatic cancer is metastatic breast cancer.

In some embodiments, the administering to the subject abraxane, doxorubicin, pamidronate disodium, anastrozole, exemestane, cyclophosphamide, epirubicin, toremifene, letrozole, trastuzumab, megestrol, novaldex, paclitaxel, docetaxel, capecitabine, goserelin acetate, or zoledronic acid.

Also provided is a method of treating a disorder, the method comprising administering to a subject in need thereof a fusion polypeptide comprising a first polypeptide comprising at least a region of a glycoprotein Ibα polypeptide and a second polypeptide comprising at least a region of an immunoglobulin polypeptide, wherein the polypeptide blocks von Willebrand Factor binding sites preventing platelet adherence allowing for a reduced amount of restrained blood flow, wherein the disorder is sickle cell anemia or thrombotic thrombocytopenia purpura.

Also provided is a method of treating metastatic cancer, the method comprising administering to a subject in need thereof a fusion polypeptide comprising a first polypeptide comprising at least a region of a glycoprotein Ibα polypeptide and a second polypeptide comprising at least a region of an immunoglobulin polypeptide, wherein the fusion polypeptide reduces the ability of tumor cells to aggregate platelets.

In some embodiments, the metastatic cancer is metastatic breast cancer.

Also provided is a method of treating a disorder, the method comprising administering to a subject in need thereof an isolated fusion polypeptide comprising a first polypeptide operably linked to a second polypeptide, wherein the first polypeptide comprises SEQ ID NO: 23 and wherein the disorder is selected from the group consisting of thrombotic thrombocytopenic purpura, infective endocarditis, metastatic cancer, and sickle cell anemia disease.

In some embodiments, the first polypeptide further comprises a signal sequence.

In some embodiments, the signal sequence comprises SEQ ID NO: 25.

In some embodiments, the second polypeptide comprises a region of a heavy chain immunoglobulin polypeptide.

The invention will be further illustrated in the following non-limiting examples.

### EXAMPLE 1

### Treatment of a Subject Having Sickle-Cell Anemia

A GPIbα-Ig containing the amino acid sequence of SEQ ID NO:5 is administered as a fusion protein by a single intravenous bolus injection (10 mg) to a patient with sickle cell anemia. Treatment with the variant ameliorates the symptoms associated with sickle cell anemia.

### EXAMPLE 2

### Treatment of a Subject Having Thrombotic Thrombocytopenic Purpura

A GPIbα-Ig containing the amino acid sequence of SEQ ID NO:5 is administered as a fusion protein by a single intravenous bolus injection (10 mg) to a patient with thrombotic thrombocytopenic purpura. Treatment with the variant ameliorates the symptoms associated with the disorder.

### EXAMPLE 3

### Treatment of a Subject Having Infective Endocarditis

A GPIbα-Ig containing the amino acid sequence of SEQ ID NO:5 is administered as a fusion protein by a single intravenous bolus injection (10 mg) to a patient with infective endocarditis. Treatment with the variant ameliorates the symptoms associated with infective endocarditis.

### EXAMPLE 4

### Treatment of a Subject Having Sickle-Cell Anemia

A GPIbα-Ig variant containing the amino acid sequence of SEQ ID NO:2 and having the amino acid substitutions Y276F, Y278F, and Y279V is administered as a fusion protein by a single intravenous bolus injection (10 mg) to a patient with sickle cell anemia. Treatment with the variant ameliorates the symptoms associated with sickle cell anemia.

### EXAMPLE 5

### Treatment of a Subject Having Thrombotic Thrombocytopenic Purpura

A GPIbα-Ig variant containing amino acids 1-290 of SEQ ID NO:2 and having the amino acid substitutions Y276F, Y278F, Y279F, K237V, and C65S is administered as a fusion protein by a single intravenous bolus injection (10 mg) to a patient with thrombotic thrombocytopenic purpura. Treatment with the variant ameliorates the symptoms associated with the disorder.

### EXAMPLE 6

### Treatment of a Subject Having Infective Endocarditis

A GPIbα-Ig variant containing the amino acid sequence of SEQ ID NO:2 and having the amino acid substitutions Y276F, Y278F, Y279V, and C65S is administered as a fusion protein by a single intravenous bolus injection (10 mg) to a patient with infective endocarditis. Treatment with the variant ameliorates the symptoms associated with infective endocarditis.

### EXAMPLE 7

### Treatment of a Subject Having Metastatic Breast Cancer

A patient diagnosed with metastatic breast cancer is treated with a GPIbα-Ig protein with the amino acid sequence of SEQ ID NO:5, which is administered as a fusion protein by a single intravenous bolus injection (10 mg). Metastatic tumor growth is evaluated and the fusion protein is readministered if necessary to inhibit tumor metastases.

### EXAMPLE 8

### Treatment of a Subject Having Metastatic Breast Cancer

A GPIbα-Ig variant containing amino acids 1-290 of SEQ ID NO:2 and having the amino acid substitutions Y276F, Y278F, Y279F, K237V, and C65S is administered as a fusion protein by a single intravenous bolus injection (10 mg) to the patient. Treatment with the combination therapy and the variant ameliorates progression of metastatic breast cancer. Metastatic tumor growth is evaluated and the fusion protein is readministered if necessary to inhibit tumor metastases.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a disorder, said method comprising administering to a subject in: need thereof a fusion polypeptide comprising a first polypeptide operably linked to a second polypeptide,
wherein said first polypeptide comprises at least a region of a glycoprotein Ibα polypeptide and said second polypeptide comprises at least a region of an immunoglobulin polypeptide,
wherein said disorder is selected from the group consisting of thrombotic thrombocytopenic purpura, infective endocarditis, metastatic cancer, and sickle cell anemia disease.

2. The method of claim 1, wherein said subject is human.

3. The method of claim 1, wherein said disorder is thrompcytopenic purpura.

4. The method of claim 3, wherein said thrombocytopenic purpura is caused by Streptococci viridians.

5. The method of claim 1, wherein said disorder is sickle cell anemia.

6. The method of claim 5, wherein sickle cell anemia results from endothelium dysfunction in said subject.

7. A method of treating a disorder, said method comprising administering to a subject in need thereof an isolated fusion polypeptide comprising a first polypeptide operably linked to a second polypeptide, wherein said first polypeptide comprises an amino acid sequence with one to 20 amino acid substitutions, deletions or insertions relative to amino acids 1-290 of a human GPIbα protein sequence (SEQ ID NO:21), wherein said first polypeptide has at least one activity selected from the group consisting of:
lower affinity binding to alpha thrombin relative to binding to alpha thrombin of a polypeptide comprising the amino acid sequence of SEQ ID NO:22;
lower aggregation relative to aggregation of a polypeptide comprising the amino acid sequence of SEQ ID NO:22; and
increased resistance to proteolysis than a polypeptide comprising the amino acid sequence of SEQ ID NO:22;
and wherein said disorder is selected from the group consisting of thrombotic thrombocytopenic purpura, infective endocarditis, metastatic cancer, and sickle cell anemia disease.

8. The method of claim 7, wherein said first polypeptide has no more than 15 substitutions, insertions, or deletions relative to the amino acid sequence of SEQ ID NO:21 or SEQ ID NO:22.

9. The method of claim 8, wherein said first polypeptide has no more than 12 substitutions, insertions, or deletions relative to the amino acid sequence of SEQ ID NO:21 or SEQ ID NO:22.

10. The method of claim 9, wherein said first polypeptide has no more than 10 substitutions, insertions, or deletions relative to the amino acid sequence of SEQ ID NO:21 or SEQ ID NO:22.

11. The method of claim 10, wherein said first polypeptide has no more than 5 substitutions, insertions, or deletions relative to the amino acid sequence of SEQ ID NO:21 or SEQ ID NO:22.

12. The method of claim 7, wherein said first polypeptide binds with lower affinity to alpha thrombin relative to binding to alpha thrombin of a polypeptide comprising the amino acid sequence of SEQ ID NO:21 or SEQ ID NO:22.

13. The method of claim 7, wherein said first polypeptide comprises at least one of the amino acid substitutions Y276F, Y278F, Y279V, or a conservative variant thereof, relative to the amino acid sequence of SEQ ID NO:22.

14. The method of claim 13, wherein said first polypeptide comprises at least two of the amino acid substitutions Y276F, Y278F, Y279V, or a conservative variant thereof, relative to the amino acid sequence of SEQ ID NO:22.

15. The method of claim 14, wherein said first polypeptide comprises the amino acid substitutions Y276F, Y278F, Y279V, or a conservative variant thereof, relative to the amino acid sequence of SEQ ID NO:22.

16. The method of claim 7, wherein aggregation of said first polypeptide is lowered relative to aggregation of a polypeptide comprising the amino acid sequence of SEQ ID NO:22.

17. The method of claim 7, wherein said first polypeptide comprises the amino acid substitution C65S, or a conservative variant thereof, relative to the amino acid sequence of SEQ ID NO:22.

18. The method of claim 17, wherein said first polypeptide is more resistant to proteolysis than a polypeptide comprising the amino acid sequence of SEQ ID NO:22.

19. The method of claim 7, wherein said first polypeptide comprises the amino acid substitution K237V, or a conservative variant thereof, relative to the amino acid sequence of SEQ ID NO:22.

20. A method of treating infective endocarditis, said method comprising administering to a subject in need thereof a fusion polypeptide comprising a first polypeptide comprising at least a region of a glycoprotein Ibα-polypeptide and a second polypeptide comprising at least a region of an immunoglobulin polypeptide, wherein said fusion polypeptide interacts with a bacterium preventing platelet aggregation.


SEQUENCE LISTING (25 sequences)

SEQ ID NO: 1 - protein, 544 aa, Artificial Sequence
  GPIb302/Ig Fusion Protein
MPLLLLLLLLPSPLHPHPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPTLGDEGDTDLYDYYPEEDTEGDKVRATRTVVKFPTKARPHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 544 aa, Artificial sequence
  GPIb302/2A-Ig Fusion Protein
MPLLLLLLLLPSPLHPHPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPTLGDEGDTDLYDYYPEEDTEGDKVAATATVVKFPTKARPHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 544 aa, Artificial sequence [sequence omitted]

SEQ ID NO: 4 - protein, 531 aa, Artificial sequence
  GPIb290-Ig Fusion Protein
MPLLLLLLLLPSPLHPHPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPTLGDEGDTDLYDYYPEEDTEGDKVRPHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 531 aa, Artificial sequence
  GPIb290-Ig
MPLLLLLLLLPSPLHPHPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQVVDVKAVTSNVASVQCDNSDKFPVYKYPGKGCPTLGDEGDTDLYDYYPEEDTEGDKVRPHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 531 aa, Artificial sequence
  GPIb290/1A-Ig Fusion Protein
MPLLLLLLLLPSPLHPHPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVAAMTSNVASVQCDNSDKFPVYKYPGKGCPTLGDEGDTDLYDYYPEEDTEGDKVRPHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 302 aa, Artificial sequence
  GPIb302
HPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPTLGDEGDTDLYDYYPEEDTEGDKVRATRTVVKFPTKA

SEQ ID NO: 8 - protein, 302 aa, Artificial sequence
  GPIb302/2A
HPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPTLGDEGDTDLYDYYPEEDTEGDKVAATATVVKFPTKA

SEQ ID NO: 9 - protein, 301 aa, Artificial sequence
  GPIb/4X
HPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQVVDVKAVTSNVASVQCDNSDKFPVKYPGKGCPTLGDEGDTDLYDYYPEEDTEGDKVAATATVVKFPTKA

SEQ ID NO: 10 - protein, 290 aa, Artificial sequence
  GPIb290
HPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVKAMTSNVASVQCDNSDKFPVYKYPGKGCPTLGDEGDTDLYDYYPEEDTEGDKVR

SEQ ID NO: 11 - protein, 290 aa, Artificial sequence
  GB1b290/2V
HPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQVVDVKAVTSNVASVQCDNSDKFPVYKYPGKGCPTLGDEGDTDLYDYYPEEDTEGDKVR

SEQ ID NO: 12 - protein, 290 aa, Artificial sequence
  GB1b290/1A
HPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQGVDVAAMTSNVASVQCDNSDKFPVYKYPGKGCPTLGDEGDTDLYDYYPEEDTEGDKVR

SEQ ID NO: 13 - protein, 16 aa, Artificial sequence
  Signal sequence
MPLLLLLLLLPSPLHP

SEQ ID NO: 14 - protein, 224 aa, Artificial sequence
  Immunoglobulin fragment
HTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1632 bp, Artificial sequence
  GPIb302/Ig
atgcctctcctcctcttgctgctcctgctgccaagccccttacacccccaccccatctgtgaggtctccaaagtggccagccacctagaagtgaactgtgacaagaggaatctgacagcgctgcctccagacctgccgaaagacacaaccatcctccacctgagtgagaacctcctgtacaccttctccctggcaaccctgatgccttacactcgcctcactcagctgaacctagataggtgcgagctcaccaagctccaggtcgatgggacgctgccagtgctggggaccctggatctatcccacaatcagctgcaaagcctgcccttgctagggcagacactgcctgctctcaccgtcctggacgtctccttcaaccggctgacctcgctgcctcttggtgccctgcgtggtcttggcgaactccaagagctctacctgaaaggcaatgagctgaagaccctgcccccagggctcctgacgcccacacccaagctggagaagctcagtctggctaacaacaacttgactgagctccccgctgggctcctgaatgggctggagaatctcgacacccttctcctccaagagaactcgctgtatacaataccaaagggcttttttgggtcccacctcctgccttttgcttttctccacgggaacccctggttatgcaactgtgagatcctctattttcgtcgctggctgcaggacaatgctgaaaatgtctacgtatggaagcaaggtgtggacgtcaaggccatgacctctaacgtggccagtgtgcagtgtgacaattcagacaagtttcccgtctacaaatacccaggaaaggggtgccccacccttggtgatgaaggtgacacagacctatatgattactacccagaagaggacactgagggcgataaggtgcgtgccacaaggactgtggtcaagttccccaccaaagcgcggccgcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggccccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 16 - DNA, 1632 bp, Artificial sequence
  GPIb302/2A-Ig
atgcctctcctcctcttgctgctcctgctgccaagccccttacacccccaccccatctgtgaggtctccaaagtggccagccacctagaagtgaactgtgacaagaggaatctgacagcgctgcctccagacctgccgaaagacacaaccatcctccacctgagtgagaacctcctgtacaccttctccctggcaaccctgatgccttacactcgcctcactcagctgaacctagataggtgcgagctcaccaagctccaggtcgatgggacgctgccagtgctggggaccctggatctatcccacaatcagctgcaaagcctgcccttgctagggcagacactgcctgctctcaccgtcctggacgtctccttcaaccggctgacctcgctgcctcttggtgccctgcgtggtcttggcgaactccaagagctctacctgaaaggcaatgagctgaagaccctgcccccagggctcctgacgcccacacccaagctggagaagctcagtctggctaacaacaacttgactgagctccccgctgggctcctgaatgggctggagaatctcgacacccttctcctccaagagaactcgctgtatacaataccaaagggcttttttgggtcccacctcctgccttttgcttttctccacgggaacccctggttatgcaactgtgagatcctctattttcgtcgctggctgcaggacaatgctgaaaatgtctacgtatggaagcaaggtgtggacgtcaaggccatgacctctaacgtggccagtgtgcagtgtgacaattcagacaagtttcccgtctacaaatacccaggaaaggggtgccccacccttggtgatgaaggtgacacagacctatatgattactacccagaagaggacactgagggcgataaggtggctgccacagcgactgtggtcaagttccccaccaaagcgcggccgcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggccccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 17 - DNA, 1632 bp, Artificial sequence
  GPIb302/4X-Ig
atgcctctcctcctcttgctgctcctgctgccaagccccttacacccccaccccatctgtgaggtctccaaagtggccagccacctagaagtgaactgtgacaagaggaatctgacagcgctgcctccagacctgccgaaagacacaaccatcctccacctgagtgagaacctcctgtacaccttctccctggcaaccctgatgccttacactcgcctcactcagctgaacctagataggtgcgagctcaccaagctccaggtcgatgggacgctgccagtgctggggaccctggatctatcccacaatcagctgcaaagcctgcccttgctagggcagacactgcctgctctcaccgtcctggacgtctccttcaaccggctgacctcgctgcctcttggtgccctgcgtggtcttggcgaactccaagagctctacctgaaaggcaatgagctgaagaccctgcccccagggctcctgacgcccacacccaagctggagaagctcagtctggctaacaacaacttgactgagctccccgctgggctcctgaatgggctggagaatctcgacacccttctcctccaagagaactcgctgtatacaataccaaagggcttttttgggtcccacctcctgccttttgcttttctccacgggaacccctggttatgcaactgtgagatcctctattttcgtcgctggctgcaggacaatgctgaaaatgtctacgtatggaagcaagtggtggacgtcaaggccgtgacctctaacgtggccagtgtgcagtgtgacaattcagacaagtttcccgtctacaaatacccaggaaaggggtgccccacccttggtgatgaaggtgacacagacctatatgattactacccagaagaggacactgagggcgataaggtggctgccacagcgactgtggtcaagttccccaccaaagcgcggccgcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggccccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 18 - DNA, 1593 bp, Artificial sequence
  GPIb290-Ig
atgcctctcctcctcttgctgctcctgctgccaagccccttacacccccaccccatctgtgaggtctccaaagtggccagccacctagaagtgaactgtgacaagaggaatctgacagcgctgcctccagacctgccgaaagacacaaccatcctccacctgagtgagaacctcctgtacaccttctccctggcaaccctgatgccttacactcgcctcactcagctgaacctagataggtgcgagctcaccaagctccaggtcgatgggacgctgccagtgctggggaccctggatctatcccacaatcagctgcaaagcctgcccttgctagggcagacactgcctgctctcaccgtcctggacgtctccttcaaccggctgacctcgctgcctcttggtgccctgcgtggtcttggcgaactccaagagctctacctgaaaggcaatgagctgaagaccctgcccccagggctcctgacgcccacacccaagctggagaagctcagtctggctaacaacaacttgactgagctccccgctgggctcctgaatgggctggagaatctcgacacccttctcctccaagagaactcgctgtatacaataccaaagggcttttttgggtcccacctcctgccttttgcttttctccacgggaacccctggttatgcaactgtgagatcctctattttcgtcgctggctgcaggacaatgctgaaaatgtctacgtatggaagcaaggtgtggacgtcaaggccatgacctctaacgtggccagtgtgcagtgtgacaattcagacaagtttcccgtctacaaatacccaggaaaggggtgccccacccttggtgatgaaggtgacacagacctatatgattactacccagaagaggacactgagggcgataaggtgcggccgcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggccccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 19 - DNA, 1593 bp, Artificial sequence
  GPIb290/2V-Ig
atgcctctcctcctcttgctgctcctgctgccaagccccttacacccccaccccatctgtgaggtctccaaagtggccagccacctagaagtgaactgtgacaagaggaatctgacagcgctgcctccagacctgccgaaagacacaaccatcctccacctgagtgagaacctcctgtacaccttctccctggcaaccctgatgccttacactcgcctcactcagctgaacctagataggtgcgagctcaccaagctccaggtcgatgggacgctgccagtgctggggaccctggatctatcccacaatcagctgcaaagcctgcccttgctagggcagacactgcctgctctcaccgtcctggacgtctccttcaaccggctgacctcgctgcctcttggtgccctgcgtggtcttggcgaactccaagagctctacctgaaaggcaatgagctgaagaccctgcccccagggctcctgacgcccacacccaagctggagaagctcagtctggctaacaacaacttgactgagctccccgctgggctcctgaatgggctggagaatctcgacacccttctcctccaagagaactcgctgtatacaataccaaagggcttttttgggtcccacctcctgccttttgcttttctccacgggaacccctggttatgcaactgtgagatcctctattttcgtcgctggctgcaggacaatgctgaaaatgtctacgtatggaagcaagtggtggacgtcaaggccgtgacctctaacgtggccagtgtgcagtgtgacaattcagacaagtttcccgtctacaaatacccaggaaaggggtgccccacccttggtgatgaaggtgacacagacctatatgattactacccagaagaggacactgagggcgataaggtgcggccgcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggccccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - DNA, 1593 bp, Artificial sequence
  GPIb290/1A-Ig
atgcctctcctcctcttgctgctcctgctgccaagccccttacacccccaccccatctgtgaggtctccaaagtggccagccacctagaagtgaactgtgacaagaggaatctgacagcgctgcctccagacctgccgaaagacacaaccatcctccacctgagtgagaacctcctgtacaccttctccctggcaaccctgatgccttacactcgcctcactcagctgaacctagataggtgcgagctcaccaagctccaggtcgatgggacgctgccagtgctggggaccctggatctatcccacaatcagctgcaaagcctgcccttgctagggcagacactgcctgctctcaccgtcctggacgtctccttcaaccggctgacctcgctgcctcttggtgccctgcgtggtcttggcgaactccaagagctctacctgaaaggcaatgagctgaagaccctgcccccagggctcctgacgcccacacccaagctggagaagctcagtctggctaacaacaacttgactgagctccccgctgggctcctgaatgggctggagaatctcgacacccttctcctccaagagaactcgctgtatacaataccaaagggcttttttgggtcccacctcctgccttttgcttttctccacgggaacccctggttatgcaactgtgagatcctctattttcgtcgctggctgcaggacaatgctgaaaatgtctacgtatggaagcaaggtgtggacgtcgcggccatgacctctaacgtggccagtgtgcagtgtgacaattcagacaagtttcccgtctacaaatacccaggaaaggggtgccccacccttggtgatgaaggtgacacagacctatatgattactacccagaagaggacactgagggcgataaggtgcggccgcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggccccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 21 - protein, 290 aa, Artificial sequence; identical to SEQ ID NO: 10
  fragment of human glycoprotein Ib chain

SEQ ID NO: 22 - protein, 290 aa, Artificial sequence; identical to SEQ ID NO: 11
  GPIb2Vvariant

SEQ ID NO: 23 - protein, 307 aa, Artificial sequence
  Fragment of immunoglobulin
QATEYEYLDYDFLPETEPPICEVSKVASHLEVNCDKRNLTALPPDLPKDTTILHLSENLLYTFSLATLMPYTRLTQLNLDRCELTKLQVDGTLPVLGTLDLSHNQLQSLPLLGQTLPALTVLDVSFNRLTSLPLGALRGLGELQELYLKGNELKTLPPGLLTPTPKLEKLSLANNNLTELPAGLLNGLENLDTLLLQENSLYTIPKGFFGSHLLPFAFLHGNPWLCNCEILYFRRWLQDNAENVYVWKQVVDVKAVTSNVASVQCDNSDKFPVYKYPGKGCPTLGDEGDTDLYDYYPEEDTEGDKVR

SEQ ID NO: 24 - protein, 16 aa, Artificial sequence
  Signal peptide
MPLLLLLLLLPSPLHP

SEQ ID NO: 25 - protein, 41 aa, Artificial sequence
  Signal peptide
MPLQLLLLLILLGPGNSLQLWDTWADEAEKALGPLLARDRR